(19) United States
(12) Patent Application Publication
(10) Pub. No.: US 2008/0154352 A1
(43) Pub. Date: Jun. 26, 2008

(54) Stent Balloon Assembly and Method of Making Same

(75) Inventor: Justin Goshgarian, Santa Rosa, CA (US)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 11/614,217

(22) Filed: Dec. 21, 2006

(51) Int. Cl. A61F 2/84; B29C 49/20

(52) U.S. Cl. 623/1.11; 264/535

(57) ABSTRACT

A method of manufacturing a stent balloon assembly includes molding a thin section in a frusto-conical portion of a balloon, and placing a stent over a stent engaging portion of the balloon when the balloon is in an unexpanded configuration. The stent engaging portion extends from the frusto-conical portion. The method also includes heating the balloon to a temperature above the glass transition temperature of the balloon, and pressurizing the balloon while the temperature is above the glass transition temperature to create a pillow from the thin section of the frusto-conical portion. The pillow protrudes outwardly relative to the stent to prevent the stent from moving in an axial position relative to the balloon.

### BACKGROUND OF THE INVENTION

1. Field of the Invention

The present invention generally relate to intraluminal stenting, and in particular to balloon catheter having a stent retention portion for minimizing axial movement of a stent mounted on the balloon catheter as the stent is delivered to the targeted site.

2. Description of Related Art

Intraluminal stenting is useful in treating tubular vessels in the body that are narrowed or blocked and it is an alternative to surgical procedures that intend to bypass such an occlusion. When used in endovascular applications, the procedure involves inserting a prosthesis into an artery and expanding it to prevent collapse of the vessel wall.

Percutaneous transluminal angioplasty (PTCA) is used to open coronary arteries, which have been occluded by a build-up of cholesterol fats or atherosclerotic plaque. Typically, a guide catheter is inserted into a major artery in the groin and is passed to the heart, providing a conduit to the ostia of the coronary arteries from outside the body. A balloon catheter and guidewire are advanced through the guiding catheter and steered through the coronary vasculature to the site of therapy. The balloon at the distal end of the catheter is inflated, causing the site of the stenosis to widen. Dilation of the occlusion, however, can form flaps, fissures or dissections, which may threaten, re-closure of the dilated vessel. Implantation of a stent can provide support for such flaps and dissections and thereby prevent reclosure of the vessel. Reducing the possibility of restenosis after angioplasty may reduce the likelihood that a secondary angioplasty procedure or a surgical bypass operation will be needed.

A plastically deformable stent can be implanted during an angioplasty procedure by using a balloon catheter bearing the compressed or “crimped” stent, which has been loaded onto the balloon. The stent radially expands into contact with the body lumen as the balloon is inflated, thereby forming a support for the lumen. Deployment is effected after the stent has been introduced percutaneously, transported transluminally, and positioned at a desired location by means of the balloon catheter.

Various methods have been used to minimize movement of the stent relative to the balloon catheter as the balloon catheter is advanced through the vessel, such as covering the stent and balloon catheter with a separate sheath, and forming pillows in the balloon with excess material that is located on opposite ends of the stent, after the stent has been mounted to the balloon. However, for small diameter balloons, forming pillows in the desired locations may be difficult, because there is less material available to form such pillows. Also, for thicker balloons, forming pillows in the desired locations may be difficult, because there may be too much material to form such pillows. In addition, the configuration of the pillows should not interfere with the deployment of the stent or contact the vessel wall as the balloon is inflated during deployment of the stent.

### SUMMARY OF THE INVENTION

It is an aspect of the present invention to provide a method for manufacturing a balloon catheter having at least one pillow configured to minimize movement of the stent along the balloon in the axial direction and not interfere with the deployment of the stent at the targeted vessel site.

In an embodiment, a method of manufacturing a stent balloon assembly is provided. The method includes molding a thin section in a frusto-conical portion of a balloon, and placing a stent over a stent engaging portion of the balloon when the balloon is in an unexpanded configuration. The stent engaging portion extends from the frusto-conical portion. The method also includes heating the balloon to a temperature above the glass transition temperature of the balloon, and pressurizing the balloon while the temperature is above the glass transition temperature to create a pillow from the thin section of the frusto-conical portion. The pillow protrudes outwardly relative to the stent to prevent the stent from moving in an axial direction relative to the balloon.

It is another aspect of the present invention to provide a balloon catheter having at least one pillow that is configured to minimize movement of the stent along the balloon in the axial direction, and not interfere with the deployment of the stent at the targeted vessel site.

In an embodiment, a stent balloon assembly is provided. The stent balloon assembly includes a stent, and a balloon catheter configured to support the stent. The balloon catheter includes a shaft and a balloon connected to the shaft. The balloon includes a stent engaging portion configured to engage an interior surface of the stent, and a pillow configured to engage one of a proximal end and a distal end of the stent so as to prevent the stent from moving in an axial direction relative to the balloon when the balloon is in an unexpanded configuration. The pillow is located on a frusto-conical portion of the balloon when the balloon is in an expanded configuration.

It is a further aspect of the present invention to provide a mold for molding a balloon for a stent balloon assembly.

In an embodiment, a mold for forming an inflatable balloon that is configured to support a stent is provided. The balloon includes a stent engaging portion and a frusto-conical portion connected to the stent engaging portion. The mold includes a mold body that defines an internal mold cavity. The cavity includes a generally cylindrical surface that is constructed and arranged to form the stent engaging portion of the balloon, and a conical surface that is connected to the cylindrical surface. The conical surface is constructed and arranged to form the frusto-conical portion of the balloon. The conical surface includes a strain inducing surface that is constructed and arranged to create a thin section in the balloon along the frusto-conical portion.

The foregoing and other features and advantages of the invention will become further apparent from the following detailed description of the presently preferred embodiments, read in conjunction with the accompanying drawings. The detailed description and drawings are merely illustrative of the invention, rather than limiting the scope of the invention being defined by the appended claims and equivalents thereof.

### BRIEF DESCRIPTION OF THE DRAWINGS

Embodiments of the present invention will now be described, by way of example only, with reference to the accompanying schematic drawings in which corresponding reference symbols indicate corresponding parts, and in which:

FIG. 1 is a side view of a portion of a stent balloon assembly according to an embodiment of the invention, with the stent in an unexpanded, crimped state;

FIG. 2 is a side view of the portion of the stent balloon assembly of FIG. 1 with the stent in the expanded state in a vessel;

FIG. 3 is a side view of the balloon portion of the stent balloon assembly of FIG. 2;

FIG. 4 is a cross-sectional view of a mold for molding a balloon for the stent balloon assembly of FIG. 1 according to an embodiment of the invention, with a parison for the balloon inserted into the mold;;

FIG. 5 is a cross-sectional view of the mold of FIG. 4 after the parison has been partially radially expanded;

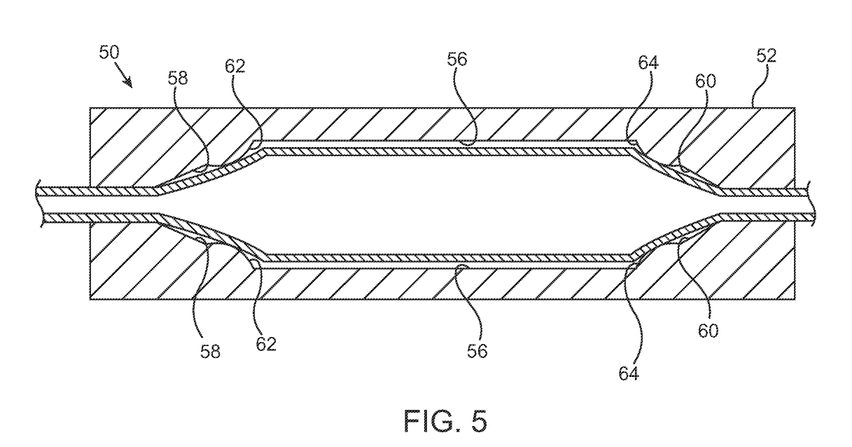

FIG. 6 is a cross-sectional view of the mold of FIG. 5 after the parison has been fully radially expanded; and

FIG. 7 is a cross-sectional view of another embodiment of a mold for molding a balloon for the stent balloon assembly of FIG. 1.

### DETAILED DESCRIPTION OF EMBODIMENTS OF THE INVENTION

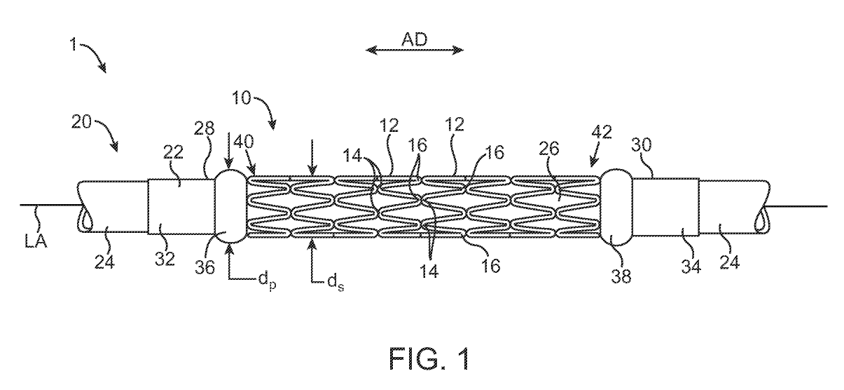

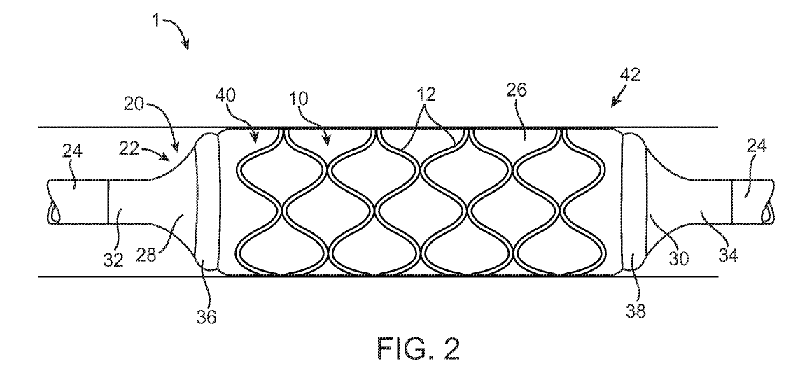

FIG. 1 shows a stent balloon assembly 1 according to an embodiment of the present invention. The stent balloon assembly includes a stent 10 that is supported by a balloon catheter 20 for delivery to a targeted site within a patient's vessel. The stent 10 may be a generally cylindrical hollow tube that is defined by a pattern comprising a plurality rings 12 having sinusoidal or zig-zag wire-forms that define a plurality of peaks 14 and a plurality of valleys 16 at opposite ends thereof. Adjacent rings 12 may be connected at selected peaks 14 of one ring and selected valleys 16 of the adjacent ring so as to form a unitary structure. The illustrated embodiment is not intended to be limiting in any way. Specifically, any alternative stent design will function in the invention, as long as the stent is plastically deformable between a compressed or crimped configuration, as shown in FIG. 1, and an expanded configuration, as shown in FIG. 2.

For example, alternative stent designs may be formed from wire-forms different from those of stent 10, including spiral zigzags, braids or a variety of other stents known to those of skill in the art of stents. Alternative stents may be made from slotted tubes or from perforated flat sheets that are rolled up into tubes. The stents 10 may be formed of biocompatible metal, such as a stainless steel alloy, a refractory metal (e.g. tungsten or tantalum), or a precipitation hardenable alloy (e.g. MP35N or PH 455). Other metal combinations are also possible, such as one metal plated with another metal for improvements in biocompatibility and/or radiopacity. Biocompatible thermoplastic or thermoset polymers are also possible alternative materials for the stent 10. The stent 10 may also incorporate any of a variety of coatings, as may be desired for enhanced friction or slipperiness, or for pharmaceutical reasons, such as for improved resistance to formation of blood clots, or reduction of arterial restenosis.

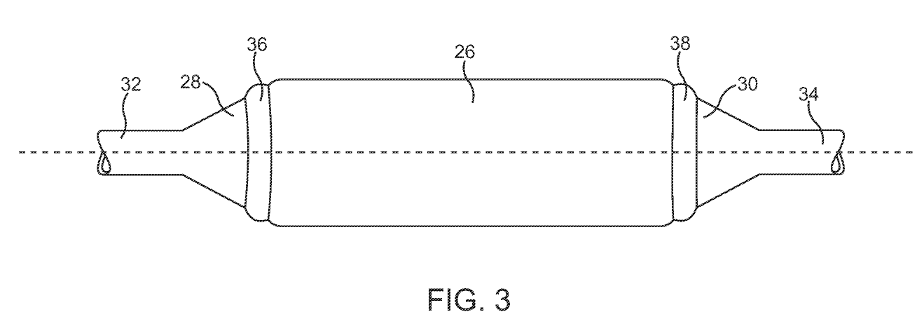

As shown in FIG. 3, the balloon catheter 20 includes a balloon 22 that is connected to at least one shaft 24. The balloon 22 includes a stent engaging portion 26 that is configured to engage an interior surface of the stent 10, as shown in FIG. 1. The stent engaging portion 26 may be generally cylindrical in shape and may be centrally located between a proximal frusto-conical portion 28 and a distal frusto-conical portion 30, when the balloon 20 is expanded, as shown in FIG. 3. The proximal and distal frusto-conical portions 28, 30 terminate in proximal and distal necks 32, 34, respectively, which are adapted to be mounted on the shaft 24, which is shown in FIGS. 1 and 2. The transitions between the stent engaging portion 26, the frusto-conical portions 28, 30, and the proximal and distal necks 32, 34 may be rounded or have a radius, rather than the sharp delineations shown in FIG. 3.

As illustrated in FIGS. 1-3, the balloon 22 also includes a proximal pillow 36 and a distal pillow 38. The proximal pillow 36 is located in the proximal frusto-conical portion 28, and is configured to engage a proximal end 40 of the stent 10 when the stent 10 mounted to the unexpanded balloon 22 in a crimped state. Similarly, the distal pillow 38 is located in the distal frusto-conical portion 30, and is configured to engage a distal end 42 of the stent 10 when the stent 10 is mounted to the unexpanded balloon 22 in the crimped state. The pillows 36, 38 are configured to prevent the crimped stent 10 from moving in an axial direction AD and a direction opposite the axial direction, respectively, relative to the balloon 22 when the balloon 22 is in an unexpanded configuration, as shown in FIG. 1. In an embodiment, a maximum diameter of the balloon at each pillows 36, 38, represented by dp in FIG. 1, is greater than an outer diameter of the crimped stent 10, represented ds, by less than about 0.010 inch. Such a difference in diameters allows the stent balloon assembly 1 to maintain a low profile, while preventing axial movement of the stent 10 relative to the balloon 22 as the stent 10 is delivered to the targeted site within the vessel. Of course, in other embodiments, the difference between dp and ds may be equal to or greater than about 0.010 inch. The actual difference between dp and ds may depend on the specific application and balloon size. For example, the difference between dp and ds may be smaller for small diameter balloon and larger for thick balloons.

When the balloon 22 is expanded, as shown in FIG. 2, because the pillows 36, 38 are located in the frusto-conical portions 28, 30 of the balloon 22, the pillows 36, 38 move away from the stent 10 such that they do not interfere with the deployment of the stent 10 in the vessel. In addition, the pillows 36, 38 do not contact the vessel. This may allow a more smooth deployment of the stent 10 and removal of the balloon catheter 20 once the stent 10 has been fully expanded.

The balloon 22 may generally be molded by the same processes used for dilation balloons, such as angioplasty balloons, or for stent delivery balloons. In general, all such balloons are made from thermoplastic polymers, including but not limited to polyvinyl chloride, polyolefin (e.g. polyethylene, irradiated polyethylene, polyethylene ionomer, polypropylene), polyester (e.g. polyethylene terephthalate), polyamide (e.g. nylon), polyurethane, ethylene-vinyl acetate, thermoplastic elastomer, and other polymers that can be biaxially oriented to impart strength and from block copolymers (e.g. polyethylene block amide, polyether block amide (PEBAX®), as well as blends and multi-layered combinations of the above-mentioned polymers. Dilatation balloons may also be made from blends that include liquid crystal polymers.

Certain polymeric materials that have been formed with a given shape may be subsequently processed to impart an even higher strength by stretching. During stretching, the molecular structure of the polymer is oriented so that the strength in that direction is higher. In a typical process of making a balloon, a polymer such as a nylon, a polyethylene block amide, or a polyurethane, for example, is first extruded into a tubular parison or preform. The parison is subsequently heated to a temperature at which it softens. By pressurizing, or blowing the parison from inside and applying axial tension, circumferential and longitudinal stretching of the parison will form a biaxially oriented balloon.

The balloon-forming step should be performed above the glass transition temperature but below the melting temperature of the base polymer material. For polymer blends and other polymer combinations, such as block copolymers, the blowing temperature should be above the highest glass transition exhibited by the material. The radial expansion and axial stretch step or steps may be conducted simultaneously, or depending upon the polymeric material of which the parison is made, following whatever sequence is necessary to form a balloon.

To create high strength, thin walled balloons, it may be desired to stretch the thermoplastic material close to its elastic limit during processing. At the end of the balloon-making process, a heat setting step may be added, wherein heat and stretching are applied to the molded balloon. The conditions of the heat setting step may be the same as or different from those used to initially form the balloon. The process of axial stretching and radial expansion may be referred to as stretch blow molding.

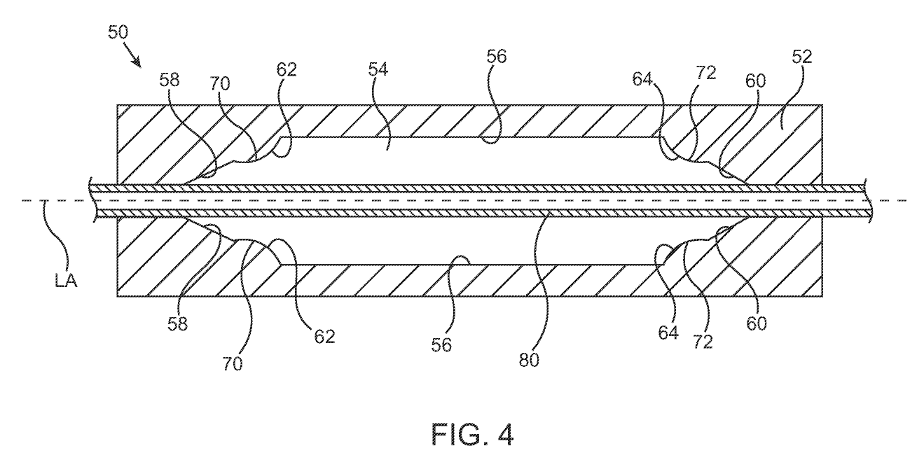

When stretch blow molding is carried out in a mold, a balloon of a predetermined shape and size may be made. To simplify mold fabrication and the removal of formed balloons, balloon molds are commonly split along one or more transverse planes, or they may be divided along a longitudinal axis. For example, FIG. 4 illustrates a balloon mold 50, which includes a mold body 52 that is split along a longitudinal axis LA. The mold body 52 defines an internal mold cavity 54 that defines the shape of the expanded balloon 22. A pressure source is connected to the mold 50 such that a pressurized gas may be supplied to axially stretch, as well as radially expand, a balloon parison. A heater and a cooler may also be connected to the mold to heat and cool the mold 50, respectively. The pressure source, heater, and cooler are well-known in the art, and no specific variants thereof are critical to practicing the instant invention. As such, they will not be described in further detail herein.

As illustrated in FIG. 4, the internal mold cavity 54 includes a generally cylindrical surface 56 that is constructed and arranged to form the stent engaging portion 26 of the balloon 22, and a first conical surface 58 that is connected to the cylindrical surface 56 at one end of the cylindrical surface 56. The first conical surface 58 is constructed and arranged to form the proximal frusto-conical portion 28 of the balloon 22. The internal mold cavity 54 also includes a second conical surface 60 that is connected to the other end of the cylindrical surface 56. The second conical surface 60 is constructed and arranged to form the distal proximal frusto-conical portion 30 of the balloon 22.

In the illustrated embodiment, the internal mold cavity 54 also includes a first strain inducing surface 62 connected to the first conical surface 58, and a second strain inducing surface 64 connected to the second conical surface 60. The strain inducing surfaces 62, 64 are each configured to induce a higher level of strain in a portion of the respective frusto-conical portions 28, 30 of the balloon 22 relative to the remaining portion of the frusto-conical portions 28, 30 as the balloon 22 is being formed. Such a configuration forms a corresponding thin section 66, 68 in the frusto-conical portions 28, 30. The thin sections 66, 68 become the pillows 36, 38 after the balloon 22 has been deflated, the stent 10 has been crimped onto the deflated balloon 22, and the balloon 22 is subjected to a suitable level of heat and pressure to form the pillows 36, 38, as discussed in further detail below.

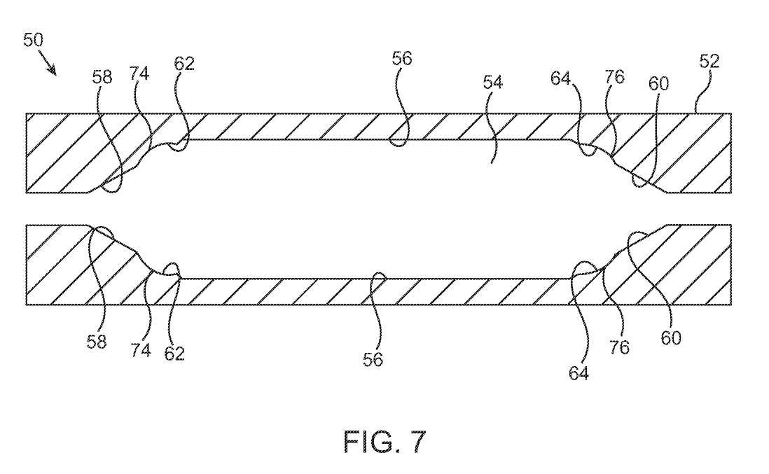

In the embodiment illustrated in FIG. 4, the strain inducing surfaces 62, 64 are each defined by a respective protrusion 70, 72 that extends from the respective first and second conical surfaces 58, 60 of the internal mold cavity 54. The protrusions 70, 72 may be integrally formed with the first and second conical surfaces 58, 60, or may be provided by separate rings (not shown) that are attached to the first and second conical surfaces 58, 60. In the embodiment of the mold 50 illustrated in FIG. 7, the strain inducing surfaces 62, 64 are each defined by a respective recess 74, 76 in the respective first and second conical surfaces 58, 60 of the internal mold cavity 54. The recesses 74, 76 may each be in the form of a continuous groove. The illustrated embodiments are not intended to be limiting in any way.

Of course, other means may be used to create the thin sections 66, 68 in the balloon 22. For example, a particular heat profile may be used during the balloon molding process that will induce more stretching in the targeted areas for the thin sections. Specifically, additional heat may be focused on the targeted areas, which will change the stretch characteristics of those areas to the extent that the thin sections 66, 68 will be created as the balloon 22 is formed in the mold 50.

In an embodiment, the balloon 22 may be formed with the following process. First, the mold 40 is provided. The mold 40 may be created by forming a material, into the desired shape of the internal mold cavity 54. The material may be glass, or glass mixed with a suitable metal, including but not limited to titanium, aluminum, and bronze. In some embodiments, separate inserts made of the same or a different material as the mold may be placed in the mold to increase the strain in the frusto-conical portions of the balloon.

A tubular balloon parison 80 may be extruded or molded from the materials listed above by known methods. Once formed, the tubular parison 80 may be heated to a temperature above the glass transition temperature of the parison material, and placed within the mold 50 such that one parison end 82 extend out of one end of the mold 50, and the other parison end 84 extends from the other end of the mold 50, when the mold 50 is in the closed position.

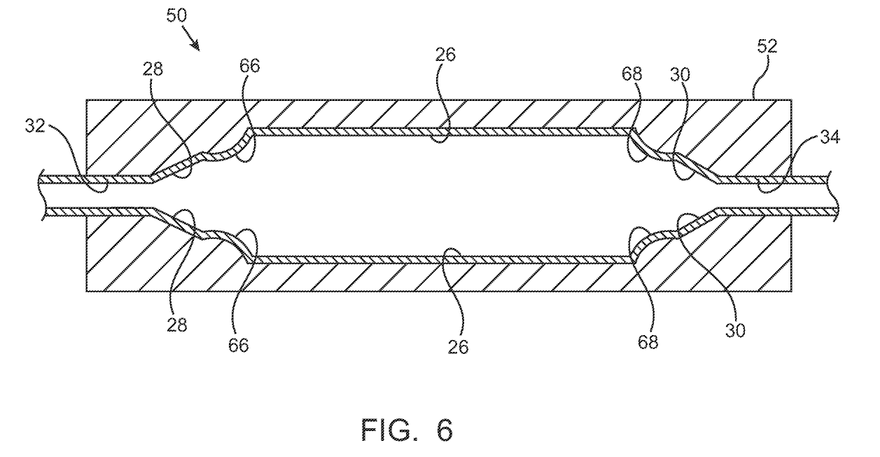

After the mold has been closed, selected pressure and axial tension may be applied to the parison 80. In an embodiment, the parison 80 may be subjected to axial tension (i.e., may be stretched longitudinally) prior to being radially expanded, so that a reduced diameter section is formed in the parison 80 prior to radial expansion. In another embodiment, the axial tension may be applied at the same time pressure is applied. In response to pressure being applied to the softened parison 80, the parison 80 expands within the mold 50, as shown in FIGS. 6 and 7, so that it contacts the internal surfaces 56, 58, 60, 62, 64 of the mold 50 that are described above. Thus, the balloon 22 is blow molded into the internal cavity 54 of the mold 50.

As shown in FIG. 7, due to the protrusions 70, 72 in the internal mold cavity 54, and the conformity of the parison 80 to the internal mold cavity 54, the thin sections 66, 68 are formed in the balloon 22. Similarly, due to the recesses 74, 76 in the internal mold cavity 54 of the mold 50 illustrated in FIG. 8, the thin sections 66, 68 are formed in the balloon 22 as the parison 80 conforms to the internal mold cavity 54. In an embodiment, the thin sections 66, 68 have average thicknesses that are about equal to an average thickness of the stent engaging portion 26 of the balloon 22, which is less than the average thicknesses of the proximal and distal frusto-conical portions 28, 30 of the balloon 22.

Although each of the protrusions 70, 72 and recesses 74, 76 are defined by a radius, it is understood that the actual value of the radius and the location of the center point of the radius may vary according to desired size and location of the resulting pillow 36, 38. Also, in some embodiments, the protrusions 70, 72 and recesses 74, 76 may have a shape that is different from the shapes illustrated in the Figures. The illustrated embodiments are not intended to be limiting in any way and are merely provided as examples of embodiments of the present invention.

In another embodiment, a heat profile may be used when heating the parison 80 so that a greater amount of heat is applied to selected portions of the parison as compared to the remainder of the parison. For example, additional heat may be applied to the corresponding portions of the parison that will form the thin sections 66, 68 of the frusto-conical portions 28, 30 of the balloon 22. Because such parison portions are at a higher temperature above the glass transition temperature than the remaining parison, the strain rate will be higher for the same application of stress, which will create a localized area of greater strain, thereby forming the thin section. The heat profile may be used with or without the embodiments of the mold discussed above.

Once the balloon 22 is blow molded, the balloon 22 may be subjected to additional heat, which may increase the burst strength to the balloon 22. Such a step may be referred to as heat setting or annealing. The additional heat may be applied while the balloon 22 is still in the mold 50 by heating the mold 50 to a temperature at which the balloon material will crystallize rapidly. In some embodiments, the balloon 22 may be removed from the mold 50 and placed in another mold (not shown) in order for the heat setting step to take place.

After the balloon 22 is cooled to a temperature that is below the glass transition temperature of the material contained therein, and after releasing any remaining pressure applied to balloon 22, the mold 50 may be opened, and the deflated balloon 22 may be removed from the mold 50. The stent 10 may then be placed over the balloon 22 in its deflated state so that the stent 10 is in contact with the stent engaging portion 26 of the balloon 22. Once the stent 10 is properly positioned on the balloon 22, e.g., is in contact with the stent engaging portion 26, the balloon 22 may be slightly pressurized so as to allow the thin sections 66, 68 to extend outward from the rest of the balloon 22 so as to form the pillows 36, 38 on opposite ends of the stent 10, as discussed above and shown in FIG. 1. The pillows 36, 38 are configured to hold the stent 10 on the balloon 22 so that the stent 10 will not slide in the axial direction AD along the balloon 22 as the stent 10 is delivered to the targeted site within the vessel.

To mount the balloon 22 onto the shaft 24, the proximal and distal necks 32, 34 of the balloon 22 are typically trimmed to a desired length. The balloon 22 may then be slid over the shaft 24, and the necks 32, 34 may then be bonded to the shaft 24 with an adhesive, thermal bonding, laser bonding, or any other suitable technique that is well-known to those skilled in the art of balloon catheters. Finally, the balloon 22 and stent 10 may be crimped about the shaft 24, with the stent 10 being plastically deformed into a compressed configuration, thereby trapping the balloon 22 between the stent 10 and the shaft 24.

When the stent balloon assembly 1 is inflated in a patient's treatment site, it will assume the substantially pre-molded, expanded configuration, as shown in FIG. 2. Because the stent 10 will be plastically deformed into the expanded configuration against the patient's vessel wall, deflation of the balloon 22 will disengage it from the stent 10, which will remain implanted in the patient's vessel.

While the invention has been particularly shown and described with reference to the embodiments and methods described above, it will be understood by those skilled in the art that various changes in form and detail may be made therein without departing from the spirit and scope of the invention.

What is claimed is:

1. A method of manufacturing a stent balloon assembly, the method comprising:
molding a thin section in a frusto-conical portion of a balloon;
placing a stent over a stent engaging portion of the balloon when the balloon is in an unexpanded configuration, the stent engaging portion extending from the frusto-conical portion;
heating the balloon to a temperature above the glass transition temperature of the balloon; and
pressurizing the balloon while the temperature is above the glass transition temperature to create a pillow from the thin section of the frusto-conical portion, the pillow protruding outwardly relative to the stent to prevent the stent from moving in an axial direction relative to the balloon.

2. The method according to claim 1, wherein the thin section is molded in a location in the frusto-conical portion such that when the balloon is expanded within a vessel, the pillow does not contact the vessel.

3. The method according to claim 1, wherein said molding comprises providing a mold defining an internal cavity having a generally cylindrical surface, and a conical surface connected to the cylindrical surface;
heating the balloon parison;
inserting a balloon parison in the mold; and
pressurizing the parison so that the parison stretches in a radial direction until the parison engages and conforms with the mold to thereby form the balloon, wherein the thin section is subjected to approximately the same strain as the stent engaging portion.

4. The method according to claim 3, further comprising stretching the parison in a longitudinal direction.

5. The method according to claim 3, wherein the mold comprises a protruding surface that extends from the conical surface near the cylindrical surface, the protruding surface being configured to create additional strain in part of the frusto-conical portion of the balloon relative to the remainder of the frusto-conical portion to form the thin section.

6. The method according to claim 5, wherein the protruding surface comprises a ring extending from the conical surface.

7. The method according to claim 6, wherein the ring is integral with the conical surface.

8. The method according to claim 3, wherein the mold comprises a recessed surface in the conical surface near the cylindrical surface, the recessed surface being configured to create additional strain in part of the frusto-conical portion of the balloon relative to the remainder of the frusto-conical portion to form the thin section.

9. The method according to claim 8, wherein the recessed surface is a groove.

10. The method according to claim 3, wherein said heating comprises providing heat to the parison in a profile so that a portion of the parison is heated to a higher temperature than the remainder of the parison, the portion being heated to the higher temperature corresponding to the thin section of the balloon.

11. The method according to claim 3, further comprising heat setting the balloon.

12. The method according to claim 1, further comprising molding a second thin section in a second frusto-conical portion of the balloon such that when the balloon is pressurized, the second thin section protrudes outward as a second pillow relative to the stent to prevent the stent from moving in a direction opposite the axial direction with the second protrusion.

13. The method according to claim 1, wherein said molding comprises blow molding.

14. The method according to claim 1, wherein the thin section has an average thickness that is about the same as an average thickness as the stent engaging portion.

15. A stent balloon assembly comprising:
a stent; and
a balloon catheter configured to support the stent, the balloon catheter comprising a shaft and a balloon connected to the shaft at proximal and distal ends thereof, the balloon comprising
a stent engaging portion configured to engage an interior surface of the stent; and
a pillow configured to engage one of a proximal end and a distal end of the stent so as to prevent the stent from moving in an axial direction relative to the balloon when the balloon is in an unexpanded configuration, the pillow being located on a frusto-conical portion of the balloon when the balloon is in an expanded configuration.

16. The stent balloon assembly according to claim 15, wherein the balloon further comprises a second pillow configured to engage the other of the proximal end and the distal end of the stent, so as to prevent the stent from moving in an direction opposite said axial direction, the second pillow being located on a second frusto-conical portion of the balloon when the balloon is in the expanded configuration.

17. The stent balloon assembly according to claim 15, wherein the pillow is created as a thin section of the frusto-conical portion during molding of the balloon.

18. The stent balloon assembly according to claim 17, wherein the thin section of the balloon has about the same strain as the stent engaging portion of the balloon.

19. The stent balloon assembly according to claim 17, wherein the thin section has an average thickness of about the same average thickness of the stent engaging portion.

20. The stent balloon assembly according to claim 15, wherein the diameter of the balloon assembly at the pillow is less than or equal to about 0.010 inch greater than the diameter of the stent when the balloon is in the unexpanded configuration.

21. The stent balloon assembly according to claim 15, wherein the balloon comprises nylon.

22. The stent balloon assembly according to claim 15, wherein the balloon comprises polyurethane.

23. The stent balloon assembly according to claim 15, wherein the balloon comprises polyether block amide.

24. A mold for forming an inflatable balloon that is configured to support a stent, the balloon comprising a stent engaging portion and a frusto-conical portion connected to the stent engaging portion, the mold comprising:
a mold body defining an internal mold cavity, the cavity comprising a generally cylindrical surface constructed and arranged to form the stent engaging portion of the balloon, and a conical surface connected to the cylindrical surface, the conical surface being constructed and arranged to form the frusto-conical portion of the balloon, the conical surface comprising a strain inducing surface constructed and arranged to create a thin section in the balloon along the frusto-conical portion.

25. The mold according to claim 24, wherein the strain inducing surface comprises a protruding surface extending from the conical surface near the cylindrical surface.

26. The mold according to claim 25, wherein the protruding surface is integral with the conical surface.

27. The mold according to claim 24, wherein the strain inducing surface comprises a recessed surface in the conical surface near the cylindrical surface.

28. The mold according to claim 24, wherein the mold body comprises glass.

29. The mold according to claim 28, wherein the mold body comprises glass and titanium.

30. The mold according to claim 24, wherein the cavity further comprises a second conical surface connected to the cylindrical surface, the second conical surface being located on an opposite side of the cylindrical surface as the conical surface and being constructed and arranged to form a second frusto-conical portion of the balloon.

31. The mold according to claim 30, wherein the second conical surface comprises a strain inducing surface constructed and arranged to create a second thin section in the balloon along the second frusto-conical portion.
